(19) United States
(12) Patent Application Publication
(10) Pub. No.: US 2003/0129673 A1
(43) Pub. Date: Jul. 10, 2003

(54) Test apparatus for the detection of pharmacologically active compounds from saliva

(75) Inventors: Ulrich Schwarz, Reinfeld (DE); Matthias Werner Engel, Ahrensburg (DE); Jin Po Lee, Carlsbad, CA (US)

Correspondence Address: FOLEY & LARDNER, P.O. BOX 80278, SAN DIEGO, CA, 92138-0278, US

(73) Assignee: Unlimited Pharmaceutical Deutschland GmBH

(21) Appl. No.: 10/286,453

(22) Filed: Nov. 1, 2002

(30) Foreign Application Priority Data
Nov. 2, 2001 (DE) 10153925

(51) Int. Cl. G01N 33/53; G01N 33/542; C12M 1/34

(52) U.S. Cl. 435/7.9; 435/287.2

(57) ABSTRACT

The present invention provides a test apparatus and methods for the detection of pharmacologically active compounds from body fluids, in particular from saliva. The invention test apparatus prevents the aqueous solution of the analyte(s) from flowing underneath the nitrocellulose membrane and ensures that even the smallest amounts of the aqueous solution of the analyte(s) are collected and pulled into and through the test strip by capillary action. The invention test apparatus provides sensible and rapid detection of the analyte(s) in the aqueous solution.

### CROSS-REFERENCE TO RELATED PATENT APPLICATION

This application claims priority to German Patent Application No. 101 53 925, entitled “Saliva Sampler”, filed Nov. 2, 2001, still pending.

### FIELD OF THE INVENTION

The invention is related to a rapid test apparatus for the detection of pharmacologically active compounds from body fluids, particularly from saliva.

### BACKGROUND OF THE INVENTION

Immunoassays, which are based on the specificity of immunological reactions and especially in primarily antigen and antibody reactions, represent sensitive detection methods for pharmacologically active compounds in body fluids. For qualitative or quantitative evaluation, it is necessary to mark one of the reaction partners with a readily detectable and measurable indicator substance, such as radioactive isotopes, enzymes, dyestuffs or fluorescent dyestuffs. These are usually solid phase tests in which the marker bound to the solid phase is measured. Solid phases which are used are microtitre plates, as in ELISA tests, or solid carriers, of which nitrocellulose has proved to be particularly suitable. Solid phase tests are constructed so that a solid carrier is divided into various sections.

The liquid aqueous medium to be analysed is applied at the starting section of the carrier, or to a separate collecting part, and the liquid is then drawn through this completely by capillary action of the carrier with excess liquid being rinsed off or collected in a liquid trap. The solid carrier is provided either with a marked antigen or marked antibody in the first section, so that upon addition of the substance to be detected (which is in solution), an antigen-antibody reaction occurs, and it is possible for the marker to be coupled either to the antigen or to the antibody. The antigen-antibody reaction product then migrates with the liquid into a second section, which contains either an immobilized antigen or an immobilized antibody with which the product from the substance to be detected and antigen or antibody reacts in a competitive reaction or, if the test is a sandwich test, undergoes a double reaction. As a result, a concentration of the marker occurs in this region, which can then be perceived qualitatively or quantitatively using an appropriate measuring apparatus or, if dyestuffs which adsorb in the visible range are employed as markers, with the dye. Marked antigens or antibodies which are employed in excess and are therefore not expended can optionally migrate further into a third section where they can then be bonded (for example, by antigens or antibodies of different specificities) and thus can be employed as a control.

Solid phase immunoassays in the competitive or sandwich technique are known in the art. Such tests are described in detail, for example, in EP-A1 0 284 232 or in EP-B1 0 291 194.

Immunoassays are carried out for the detection of pharmacologically active compounds in urine or serum, although many substances would be detectable via saliva. Analyses from urine or blood require active participation of the subject. If the subject refuses participation, analyses cannot be carried out at all; or it can be carried out only in accordance with legal provisions. From security forces, numerous hospitals, and doctors' practices, there is an interest in being able to carry out rapid tests on the basis of saliva samples, which was hitherto practically impossible.

Saliva is an aqueous secretion of which approximately 1 to 2 litres per day is produced by humans. However, in addition to a number of cations, as well as anions, saliva also comprises enzymes and, in particular, mucins, which cause a mucilaginous property of such secretions. Saliva is therefore a liquid which behaves differently from a purely aqueous solution because of its viscosity and therefore causes difficulties in analyses based on the principle of chromatography, which can lead to greatly increased reaction times and uncertain or false results. The viscosity of saliva can vary, not only from person to person, but also according to stimulation or medicaments. In addition, the concentration of pharmacologically active substances or their metabolites in saliva is usually lower than the concentration found in other body fluids, such as urine.

Citation of documents herein is not intended as an admission that any of the documents cited herein are pertinent prior art or an admission that the cited documents are considered material to the patentability of the claims of the present application. All statements as to the date or contents of these documents are based on the information available to the applicant and does not constitute any admission as to the correctness of the dates or contents of these documents.

### SUMMARY OF THE INVENTION

The invention is based on the object of developing a test apparatus for the detection of pharmacologically active substances in saliva which can be employed as a rapid test apparatus and leads to reliable results.

It has been found that immunoassays can also be performed on saliva if the saliva is taken up by a membrane, extracted with a buffer, and exposed to a “filtering action” by a collecting membrane. During this operation, the mucins with their complex structures and their high molecular weights are already held in this collecting membrane, so that a substantially aqueous solution of the analyte can be subjected to an immunoassay in the conventional manner.

The test apparatus, according to the invention, comprises an upper and lower section which are designed so that a prepared test strip for the analyte is held by a test strip holder in the lower section of the apparatus. The lower section has a receiving device constructed as a sample collector. The sample collector is covered with a porous collective membrane, such as a synthetic nonwoven. The collecting membrane serves to separate saliva and the aqueous extract of the drugs to be investigated. The collecting membrane is made of fibres or a nonwoven for example, but it is ensured that the collecting membrane itself does not adsorb the compounds to be detected. Polyamide is preferably employed since cotton, for example, firmly bonds with THC (tetrahydrocannabinol) and other hydrophobic compounds.

The collecting membrane lies on a receiving section which serves to adsorb the liquid into the test strip. The parts of the lower section lying underneath this receiving section are inclined downward and form an empty space between the receiving section and the plastic reinforcement of the test strip. This constructional measure means that the flow of the saliva extract is directed directly onto the nitrocellulose membrane. Without such an empty space, the saliva extract would flow only underneath the test strip by capillary forces at the connecting point between the test strip and collecting membrane.

The liquid to be tested flows on the basis of capillary action from the collecting membrane into a porous reaction cushion where the reaction between the analyte and the marked antigen or antibody takes place. The reaction complex then migrates to the porous carrier of nitrocellulose from the reaction zone, into the detection zone, and into the control zone. The excess liquid is absorbed into an adsorption membrane. The test strip itself is preferably designed so that not only one, but several analytes, can be determined at the same time, since simultaneous dependency on various addictive substances occurs relatively frequently.

With automatic reading of the test result, the reading apparatus is adjusted to the control line. The test strip is positioned and fixed accurately by the test strip holder and the distance between the detection zones or the detection lines formed are kept substantially identical. The detection lines would have a separation of approximately 5 mm with deviations of not more than +/−0.5 mm.

The upper part of the test apparatus comprises a cover which has one or more windows which allow observation of the detection zones or lines and the control zone or line. A cap is attached over the front part of the upper section. The cap is made of an adsorbent membrane which is sponge-like or which can be constructed so that it covers a cap-shaped sponge covered by the membrane. The sponge contains a buffer which serves to extract the aqueous solution of the analyte from the saliva, so that the aqueous solution of the analyte is transferred from the sponge onto the collecting membrane. The aqueous solution of the analyte is then passed on to the reaction cushion and to the test strip by capillary action.

When the saliva sample is taken, the cap is employed like a spatula for receiving the saliva from the mucous membrane of the cheek. The majority of the saliva sample is collected in the front region of the cap and complete mixing and extraction by the buffer is not ensured. The test apparatus is therefore preferably constructed so that the collecting membrane is in direct contact with the sponge containing the buffer, and the aqueous saliva extract flows into the lower section of the apparatus, which then has a machined-in capillary grid which spatially corresponds directly to the upper section and narrows funnel-shaped to the transition to the test strip. This ensures that even the smallest amounts of the aqueous solution of the analyte or analytes are collected and sucked up onto the test strip by capillary action.

In another embodiment, instead of the cap described above, which is employed like a spatula, the buffer sponge is covered by a ceramic disc with perforations which then serves as a spatula. The saliva flows through the buffer sponge and downwards through a ceramic filter plate, so that the interfering constituent of the saliva is retained and the aqueous saliva extract is employed for the analysis in the manner described.

The result of the test is observed through a control window in the upper section, if the markers lead to a visually perceptible reaction product, which is sufficient if the determination is only qualitative. If an electronic evaluation of the test strip is necessary, particularly in the case of quantitative determinations, the upper section is constructed so that it can be opened either by unfolding the upper side or by an intentional breaking point between the region of the test strip and the reaction cushion, and the test strip is removed. However, in the event of correct and accurate positioning of the test strip by test strip holders, it is not necessary, since an electronic evaluation takes place through the observation window.

The transition between the nitrocellulose test strip and the sample collector prevents saliva extract from flowing underneath the nitrocellulose membrane by capillary forces. In a preferred embodiment, the collecting membrane lies on an adsorption cushion, which is directly adjacent to the nitrocellulose membrane. The nitrocellulose membrane lies on a sheet of plastic which serves as reinforcement. A free space formed by slanting of the lower section underneath the adsorption cushion avoids the undesirable bypass of the saliva extract. The space prevents capillary forces from being active so that the saliva extract hydrogenates under the cellulose membrane, but instead collects it in the free space.

The collecting cap adjacent to the upper section comprises a part of plastic which is open at the top and is fixed to the lower section of the test apparatus, by a press fit. This cap represents a type of flat dish which is filled with a foam impregnated with the extraction buffer. The cap is made of a flexible plastic, so that the buffer is pressed onto the collecting membrane. The upper cover of the test apparatus is made of plastic with an observation window and is clipped onto the lower part of the test apparatus by a press fit. The observation window is in the correct position with certainty during evaluation by electronic apparatuses, appropriate grooves are present in the lower part in order to guarantee correct fit.

Since the test apparatus cap provided with foam is used like a spatula for collecting saliva from the mucous membrane of the cheek, the majority of the saliva is on the cap. However, since the saliva is to be completely mixed with the buffer and extracted, and the analytes to be detected are often present in saliva, only in a small amount, in a preferred embodiment, a type of funnel-shaped guide for the saliva or saliva extract is provided in the lower part of the test apparatus below the collecting device. The buffered foam is directly above the funnel-shaped depression so that after collection of the saliva sample reliable mixing of the saliva and the buffer takes place by gentle pressure on the cap, which is made of flexible plastic in its constructional parts, and the saliva extract then flows into the funnel-shaped depression.

In another preferred embodiment, which ensures that sufficient saliva is obtained during sampling, the cap with the foam is designed so that the foam is rotated during sampling and, at the same time, the saliva extract is pressed through the collecting membrane. The foam is in a perforated carrier or is connected onto a rotatable pin so that the foam is rotated in the dish-shaped depression and is gently compressed. Complete mixing of the buffer and saliva and the simultaneous flowing out of the saliva extract, practically without trace contamination are ensured. The construction of the test apparatus allows small amount of analytes to be determined, such supposed or actual abstinence from the analyte to be investigated.

### BRIEF DESCRIPTION OF THE DRAWINGS

FIG. 1 illustrates a saliva collector according to the invention.

FIG. 2 illustrates a cross-section of a particular construction at the transition from the collecting membrane to the adsorption cushion.

FIG. 3 illustrates a cross-section of a particular lower part of the apparatus.

FIG. 4 illustrates a particular upper part of the apparatus.

### DETAILED DESCRIPTION OF INVENTION

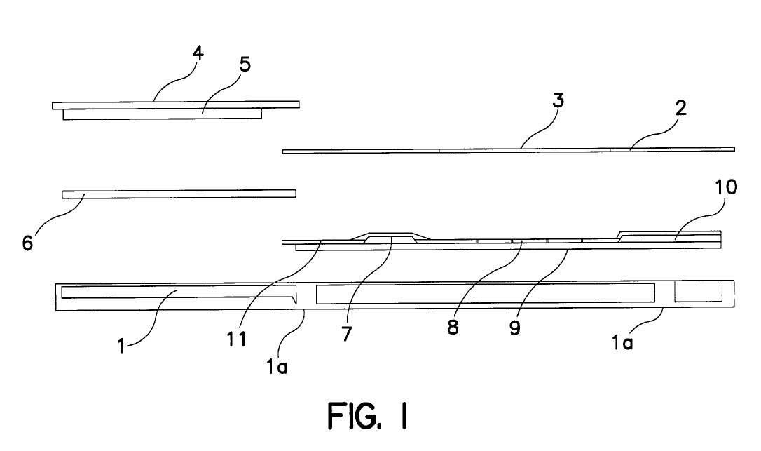

As seen in FIG. 1, the test apparatus comprises a lower section (1) of plastic with test strip holders (1a) and the upper section (2) with the observation window (3) and the collecting cap (4) with the buffered foam (5). The saliva is received and extracted by the collecting cap (4) and the buffered foam (5) is transferred to the collecting membrane (6), which is in direct capillary contact with the adsorption wick (11) and with the adsorption cushion (7). The aqueous extract with the analyte then passes by capillary action to the nitrocellulose strip (8), which is joined to a carrier (9) of plastic. Excess liquid is absorbed by the adsorption cushion (10).

When handling the test apparatus, a saliva sample is transferred with the aid of the collecting cap (4) to the buffer foam (5) so that a saliva extract with the analytes then passes to the collecting membrane (6), from where it migrates to the adsorption wick (11) and then by capillary action to the adsorption cushion (7). The reaction of the analyte with the marked reagent takes place in the adsorption cushion (7) and the reaction product then migrates to the nitrocellulose matrix (8), which is provided at predetermined points with an immobilized second reagent which reacts with the marked reagent and thus leads to an accumulation of the marker at the detection line. The cellulose matrix has several detection lines for various analytes, and a control line in which excess reagent from the adsorption cushion (7) is reacted with a second reagent of different specificity. Excess liquid can be collected in the adsorption cushion (10).

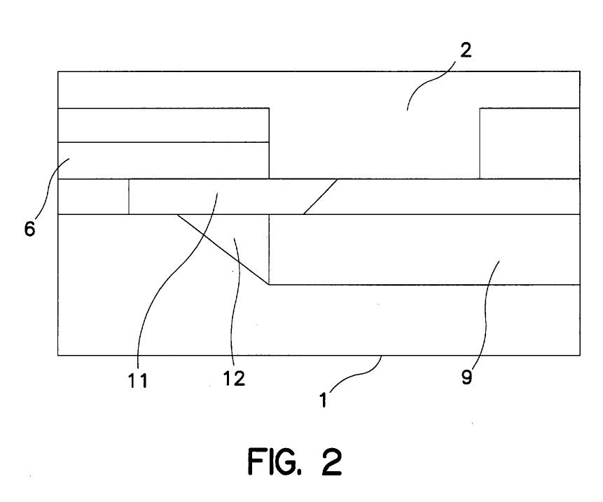

To prevent at least some of the aqueous solution migrating below the adsorption cushion or below the nitrocellulose strip (8) by capillary action present in the transition from the collecting membrane (6) to the adsorption wick (11), in a preferred embodiment of the invention, the transition space can be constructed so that the collecting membrane (6), which is between the upper section (2) and lower section (1), is in contact with the adsorption wick (11), which can be constructed as several parts (FIG. 2). The adsorption wick (11) is located on the carrier (9) of plastic. At the transition between the collecting membrane (6) and adsorption wick (11), the lower section (1) is inclined at an angle of approximately 45° up to the start of the carrier (9) of plastic, so that a free or collecting space for the saliva extract forms and an undesirable capillary action on the under-side of the wick (11) is prevented.

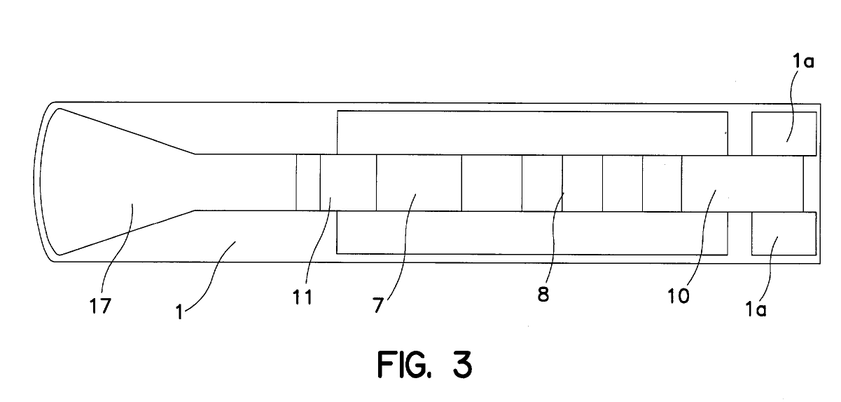

In another preferred embodiment, in order to evaluate small amounts of saliva extract, the lower section (1) with the test strip holders (1a) is constructed in the front region so that small amounts of the extract can also migrate via the adsorption wick (11) and the adsorption cushion (7) on the nitrocellulose strip (8), up to the adsorption wick (11) and the adsorption cushion (7), onto the nitrocellulose strip (8), up to the adsorption cushion for excess liquid (10), in that the front part of the collecting space in the lower section (1) contains a funnel-shaped construction 17 (FIG. 3). Small amounts of liquid are also transferred to the adsorption wick (11) by this method.

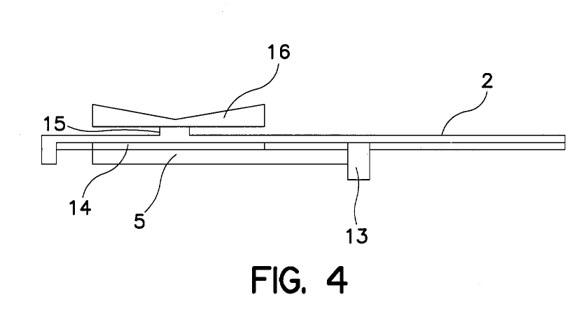

In order to achieve a reliable test result with small amounts of saliva or very highly viscous saliva, in another preferred embodiment, the upper section (2) can be constructed such that the cap (4) is provided with a support plate (14) and buffered foam (5) fixed thereon so that the support plate with the buffered foam is rotated by a rotary knob (16) with a seal (15) and is thus pressed out and the extract is passed to the saliva collector (13) (FIG. 4).

The test apparatus, according to the invention, is also designed so that it comprises several test strip holders and several test strips. For example, in addition to the saliva test, a urine test for pharmacologically active substance s and a test for the presence of such substances in the environment of the subject are carried out. For the environment test, areas which are suspected of showing traces of the pharmacological substances to be investigated are wiped with, for example, a ceramic spatula which is then rinsed off with a given small amount of water so that an aqueous solution of the analyte is present, which is then analyzed using the invention apparatus.

The present invention having been fully described, is not to be limited in scope by the specific embodiments described which are intended as single illustrations of individual aspects of the invention. Functionally equivalent methods and components are within the scope of the invention. Various modifications of the invention, in addition to those shown and described herein, will become apparent to those skilled in the art from the foregoing description and accompanying drawings. Such modifications are intended to fall within the scope of the appended claims.

The invention claimed is:

What is claimed is:

1. A test apparatus for the detection of pharmacologically active compounds in a body fluid, comprising:
an upper part comprising one or more observation windows; and
a lower part of plastic which are joined to said upper part;
wherein said upper part and said lower part are constructed as a sample collector;
wherein further said upper part comprises a collecting cap fixed with a buffered foam;
wherein further said lower part comprises test strip holders, a carrier of plastic located in between, and a nitrocellulose strip connected with an adsorption cushion and an adsorption wick, and
wherein the body fluid extract formed in said buffer foam is passed on to a collecting membrane connected to said adsorption wick.

2. The test apparatus according to claim 1, wherein said collecting cap comprises a buffered foam.

3. The test apparatus according to claim 1, wherein a separate buffered foam layer is located under said collecting cap.

4. The test apparatus according to claim 1, wherein said lower part has an inclination before said carrier of plastic in the region of the transition between said collecting membrane and said adsorption wick.

5. The test apparatus according to claim 4, wherein an angle of the inclination of said lower part is preferably 45°.

6. The test apparatus according to claim 1, wherein a collecting funnel before the transition to said adsorption wick is formed in said lower part for collecting body fluid extract.

7. The test apparatus according to claim 1, wherein said buffered foam in said collecting cap is compressed.

8. The test apparatus according to claim 7, wherein said buffered foam is compressed by a rotary knob.

9. The test apparatus according to claim 1, wherein said collecting cap comprises a ceramic disc with perforations with a buffered foam fixed to a ceramic filter plate.

10. The test apparatus according to claim 1, wherein said body fluid is saliva.

11. The test apparatus according to claim 1, wherein said body fluid is urine.

12. A test apparatus for the detection of pharmacologically active compounds in saliva, comprising:
an upper part comprising a cover having one or more observation windows; and a lower part of plastic which are joined to said upper part;
wherein said upper part and said lower part are constructed as a sample collector;
wherein further said upper part comprises a collecting cap fixed with a buffered foam;
wherein further said lower part comprises test strip holders, a carrier of plastic located in between, and a nitrocellulose strip connected with an adsorption cushion and an adsorption wick,
wherein further an inclination of said lower part is formed before said carrier of plastic in the region of the transition between a collecting membrane connected to said absorption wick; and
wherein the saliva extract formed in said buffer foam is passed on to said collecting membrane connected to said adsorption wick.

13. The test apparatus according to claim 12, wherein an angle of the inclination of said lower part is preferably 45°.

14. The test apparatus according to claim 12, wherein said collecting cap comprises a buffered foam.

15. The test apparatus according to claim 12, wherein a separate buffered foam layer is located under said collecting cap.

16. The test apparatus according to claim 12, wherein said buffered foam in said collecting cap is compressed.

17. The test apparatus according to claim 12, wherein said buffered foam is compressed by a rotary knob.

18. The test apparatus according to claim 12, wherein said collecting cap comprises a ceramic disc with perforations with a buffered foam fixed to a ceramic filter plate.

19. A test apparatus for the detection of pharmacologically active compounds in saliva, comprising:
an upper part comprising a cover having one or more observation windows; and a lower part of plastic which are joined to said upper part;
wherein said upper part and said lower part are constructed as a sample collector;
wherein further said upper part comprises a collecting cap fixed with a buffered foam;
wherein further said lower part comprises test strip holders, a carrier of plastic located in between, and a nitrocellulose strip connected with an adsorption cushion and an adsorption wick,
wherein further a collecting funnel before the transition to said absorption wick is formed in said lower part for collecting saliva extract; and
wherein the saliva extract formed in said buffer foam is passed on to a collecting membrane connected to said adsorption wick.

20. The test apparatus according to claim 19, wherein said collecting cap comprises a buffered foam.

21. The test apparatus according to claim 19, wherein a separate buffered foam layer is located under said collecting cap.

22. The test apparatus according to claim 19, wherein said buffered foam in said collecting cap is compressed.

23. The test apparatus according to claim 19, wherein said buffered foam is compressed by a rotary knob.

24. The test apparatus according to claim 19, wherein said collecting cap comprises a ceramic disc with perforations with a buffered foam fixed to a ceramic filter plate.

25. A method for the detection of pharmacologically active compounds in saliva, comprising:
sampling saliva from a subject on the test apparatus according to claim 1; and
detecting the presence or absence of pharmacologically active compound in said saliva.

26. A method for the detection of pharmacologically active compounds in saliva, comprising:
sampling saliva from a subject on the test apparatus according to claim 12; and
detecting the presence or absence of pharmacologically active compound in said saliva.

27. A method for the detection of pharmacologically active compounds in saliva, comprising:
sampling saliva from a subject on the test apparatus according to claim 19; and
detecting the presence or absence of pharmacologically active compound in said saliva.
